(19) United States
(12) Patent Application Publication
(10) Pub. No.: US 2008/0233182 A1
(43) Pub. Date: Sep. 25, 2008

(54) ORGANIC COMPOUNDS

(76) Inventors: Georg Ludwig Kis, Triboltingen (CH); Jacques Vandermander, Cagnes-Sur-Mer (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/052,016

(22) Filed: Mar. 20, 2008

(30) Foreign Application Priority Data
Mar. 23, 2007 (EP) 07104792.2
Jun. 28, 2007 (EP) 07111322.9

(51) Int. Cl. A61K 33/34; A61K 9/127; A61K 9/48; A61K 31/30; A61P 3/10; A61P 27/02

(52) U.S. Cl. 424/450; 424/630; 424/638; 424/451; 424/635; 424/632; 424/634; 514/499; 514/500

(57) ABSTRACT

The present invention relates to a method of protecting copper-sensitive compounds and/or compositions from decomposition.

The present invention relates to a method of protecting copper-sensitive compounds and/or compositions from decomposition.

Omega-3-fatty acids and a number of vitamins and other pharmaceutically effective compounds or compositions are susceptible to decomposition when in contact with copper salts. This decomposition is primarily a problem during storage of such compounds and/or compositions and in particular when water is present. Said presence of water applies also to minute amounts of water, such as humidity per se, traces of water in one of the components of a composition and/or of a compound, or from environmental humidity.

There are a number of compounds and/or compositions, which contain a copper salt as an important and hence often mandatory ingredient. In such a situation, the interaction of a copper salt and the copper sensitive compound and/or composition is controlled by the provision(s) provided in the next paragraphs, i.e. decomposition of the copper sensitive compound and/or composition is substantially inhibited or typically prevented.

In a first aspect, the problem is solved by providing the copper salt with a proper masking or coating. This masking or coating typically suppresses an interaction with said copper sensitive compound and/or composition. The copper salt is for example masked or coated with—or embedded in—gelatin or liposomes and/or both such as for example a coated copper salt is embedded in gelatin. There are other ways of protecting copper salts, e.g. by encapsulating it with mono- and di-glycerides, as for example known from the product Descote® which is commercially available.

Copper salts can form a stable complex with an appropriate cyclodextrin compound, which would typically release its free copper salt at a later stage, e.g. when deemed required e.g. upon enzymatic resolution of the complex in the gut.

Copper salts might be coated with a zwitterionic phospholipid including but not limited to phosphatidylcholine, phosphatidylserine, phosphatidylethanolamine, spingomyelin and other ceramides, as well as various other zwitterionic phospholipids.

In the enclosed examples there are a number of copper sensitive compounds which decompose if the copper is not properly masked or coated. Such a copper sensitive composition may for example contain:

### EXAMPLE 1

| Ingredients | Amount | Source |
|---|---|---|
| Vitamin C | 500 mg | Calcium ascorbate dihydrate, USP |
| Vitamin E | 400 IU | Alpha tocopheryl acetate, USP |
| Zinc | 40 mg | Zinc oxide |
| Copper | 1 mg | Cupric carbonate, basic, in acc. to Merck |
| | | Index |
| Total Omega 3 | 350 mg | Omega-3 acid triglycerides Ph.Eur. 120 mg |
| fatty acids | | (DHA:EPA ratio from 1:4, 1:3, 1:2, |
| | | 1:1, 2:1, 3:1, or 4:1) |
| Lutein | 10 mg | FloraGlo 20% natural source |
| Zeaxanthin | 2 mg | from Lutein |

### EXAMPLE 2

In another experiment, the copper, e.g. cupric carbonate, basic, is incorporated in the shell of a capsule which contains the below ingredients. Specifically, the excipients for making such a shell are for example:

| | |
|---|---|
| Gelatin | 175 bloom bovine |
| Glycerin | 99%, USP |
| Soybean oil flakes (hydrogenated), | NF |
| Soybean oil | USP, and |
| | Colorants (if required), organic |
| | and/or inorganic, e.g. Titanium |
| | dioxide USP, black iron oxide |
| | E172 and/or red iron oxide E172 |

The stability of the above composition is investigated in a setup where the copper is not masked, and in a situation wherein the copper is masked in the shell, and hence not in direct contact with the copper sensitive ingredients. The stability of the described masking/coating method correlates with the degree of decomposition of such a copper sensitive compound and/or composition.

### EXAMPLE 3

| | |
|---|---|
| 240 mg Fish oil with 70% DHA | label 160 mg Omega 3 |
| | fatty acids |
| Docosahexaeonic acid (DHA) | label 130 mg DHA |
| Eicosapentaenoic acid (EPA) as part of fish oil | label 11 mg |
| 91.5 mg Calcium ascorbate | label 60 mg Vitamin C |
| 31.343 mg d,l Alpha-tocopheryl acetate | label 20 mg Vitamin E |
| 55 mg Lutein 20% in safflower oil | label 10 mg Lutein |
| 12.447 mg Zinc oxide | label 10 mg Zinc |
| 0.45 mg Cupric carbonate basic | label 0.25 mg Copper |
| Zeaxanthin as part of Lutein | label 0.8 mg |

The above composition may contain other excipients for the production of a targeted galenic formulation, e.g. for making a shell of a capsule or the like.

### EXAMPLE 4

Stability of Omega 3 Fatty Acids in Soft Gelatin Capsules after 2 Days' Storage at 80° C. (Ambient Humidity i.e. 50-70% Relative Humidity)

(Change in the Content of the Omega 3 Fatty Acid's Components)

The assays to measure the percentage in weight of EPA and DHA after storage are carried out by gas chromatography by the standard method described in the European Pharmacopoeia 5.4.

| Omega 3 fatty acids' | Formulation with | | |
|---|---|---|---|
| components | unmasked Copper | Formulations with masked Copper | |
| : | Formulation as | Formulation as | Formulation as |
| | disclosed in example | disclosed in example | disclosed in example |
| | 3: (with Cupric | 3, wherein the Cupric | 3, however the |
| | carbonate, basic). | carbonate, basic; is | Copper being |
| | The shell is identical | replaced by | omitted from said |
| | to the shell disclosed | DESCOTE ® (coated | formulation. |
| | in example 2 but | Copper gluconate) | Copper is however in |
| | does not contain any | 2.250 mg | the shell, namely |
| | Copper | corresponding to | 0.45 mg of Cupric |
| | | 0.25 mg Copper. The | carbonate, basic |
| | | shell is identical to | (corresponding to |
| | | the shell disclosed in | 0.25 mg Copper) as |
| | | example 2 but does | disclosed in example 2 |
| | | not contain any | |
| | | Copper | |
| EPA in weight % | 5.0% decrease | 1.7% decrease | unchanged (no |
| | | | decrease) |
| DHA in weight % | No detectable | No detectable | unchanged (no |
| | change | change | decrease) |
| Other omega 3 fatty | 1.9% decrease | 0.4% decrease | unchanged (no |
| acids** than EPA | | | decrease) |
| and DHA in weight % | | | |

**Other Omega 3 fatty acids denote the sum of alpha-linolenic acid (ALA), stearidonic acid (SA), eicosatetraenoic acid (ETA) and docosapentaenoic acid (DPA).

Bibliographic Convention:

As used herein, copper or a copper salt are used interchangeably and pertain preferably Cu²⁺ but may also include Cu⁺. A copper salt comprises preferably a pharmaceutically acceptable anion such as chloride, oxide, hydroxide, gluconate, carbonate, sulfate or the like.

As used herein, an omega-3-fatty acid are mainly but not only eicosapentaenoic acid (EPA) and docosahexaenoic acid (DHA).

As used herein, the term masked, coated or embedded copper or copper salt pertains to a copper salt and a masking, coating or embedding agent, which effectively prevents a direct interaction of said copper salt with said copper sensitive compound and/or composition.

As used herein, a digestible capsule material is for example the shell material described in the above Example 2, but is in general any capsule or capsule shell or shell material being used in the state-of-the-art for pharmaceutical/dietary/nutraceutical capsules, typically being organic polymeric compositions and typically being pharmaceutically compatible (non-toxic, bio-degradable, digestible in the gut or stomach as deemed suitable).

As used herein, the term pharmaceutical composition typically refers to a food supplement composition and/or neutraceutical composition and vice versa.

In another aspect the present invention relates to the use of a masked or coated copper salt in a pharmaceutical composition for the treatment of a disease being treatable by a copper salt, e.g. age related macular degeneration (AMD) or diabetic retinopathy (DR).

The invention further relates to the use of a masked or coated copper salt in a method to stabilize a pharmaceutical composition.

The invention further relates to a storage stable pharmaceutical composition comprising an omega-3-fatty acid and a coated or masked copper salt.

The invention further relates to use a masked or coated copper salt in a method to stabilize a food supplement and/or neutraceutical composition from copper salt dependent decomposition.

The invention further relates to the use of a masked or coated copper salt in a method to stabilize a pharmaceutical composition from copper salt dependent decomposition.

The invention further relates to a drug delivery system comprising a digestible capsule material, or shell, or shell material, one or more copper sensitive component(s) such as vitamin E, lutein, or omega 3 fatty acids, and a copper salt characterized in that said copper salt is placed into the shell material of said drug delivery system.

The invention further relates to:

A digestible capsule material, in particular a gastrointestinal tract digestible capsule material, containing a masked and/or coated copper salt, preferably Copper sulfate, Copper carbonate and/or Copper gluconate, characterized in that said capsule material represent said masking and/or coating material for said copper salt;

A digestible capsule material as defined in the foregoing paragraph, wherein said copper salt is already coated with a coating, and said coating might be the same or another masking material than said capsule material;

A digestive capsule material as defined in the 2 foregoing paragraphs comprising in the fill the following ingredient in the fill vitamin C which comprises or substantially consists of calcium ascorbate, vitamin E which comprises or substantially consists of d,l alpha tocopheryl acetate, zinc which comprises or substantially consists of zinc oxide, copper which comprises or substantially consists of Copper oxide, Copper sulfate, Copper carbonate or Copper gluconate, and an omega 3 fatty acid which comprises or substantially consists of Omega-3 acid triglycerides Ph.Eur.

The use of a masked or coated copper salt, said masking being obtained in accordance to the above description, especially in the form as a digestive capsule material as described above, in a nutrient, or in a dietary supplement, in particular in a purpose as described hereinbefore, and optionally in conjunction with an AMD treatment, such as treatment with Lucentis® or Visudyne®;

The use of a masked or coated copper salt in accordance to the above description as a nutrient composition, or as a dietary supplement composition, in the prevention/treatment of AMD and/or DR.

Method of treating and/or preventing AMD and/or DR in a subject being in need thereof, comprising administering an effective amount of a nutrient composition, or a dietary supplement composition comprising masked or coated copper salt in accordance to the above description.

1-8. (canceled)

9. A digestible capsule material, containing a masked and/or coated copper salt, characterized in that said capsule material represent said masking and/or coating material for said copper salt.

10. A digestible capsule material of claim 9, wherein said copper salt is already coated with a coating, and said coating comprises the same or another masking material than said capsule material.

11. A digestive capsule material of claim 9 the 2 comprising in the fill the following ingredient in vitamin C which comprises calcium ascorbate, vitamin E which comprises d,l alpha tocopheryl acetate, zinc which comprises zinc oxide and an omega 3 fatty acid which comprises Omega-3 acid triglycerides Ph. Eur.

12-13. (canceled)

14. Method of treating and/or preventing AMD and/or DR in a subject being in need thereof, comprising administering an effective amount of a nutrient composition, or a dietary supplement composition comprising a masked or coated copper salt in the form of a digestive capsule according to claim 9.

15. A method of treating and/or preventing a disease being treatable and/or preventable by administration of a copper salt, comprising administering an effective amount of a pharmaceutical composition comprising a masked or coated copper salt.

16. The method of claim 15, wherein said disease is age related macular degeneration or diabetic retinopathy.

17. The method of claim 15, wherein said pharmaceutical composition comprises one or more copper sensitive compounds such as an omega-3-fatty acid, vitamin C, vitamin E, lutein and/or zeaxanthin.

18. The method of claim 15, wherein said copper is coated with gelatin, a liposome, or a mono- and di-glyceride.

19. The method of claim 15, wherein said copper is copper gluconate, copper oxide, copper carbonate or copper chloride.

20. The method of claim 15, wherein said coating is a zwitterionic phospholipid including but not limited to phosphatidylcholine, phosphatidylserine, phosphatidylethanolamine, spingomyelin and other ceramides, as well as various other zwifterionic phospholipids.

21. The method of claim 15, wherein said copper is a copper cyclodextrin complex.

22. A method of stabilizing a pharmaceutical composition from copper salt dependant decomposition, comprising the addition of a masked or coated copper salt to said pharmaceutical composition.

23. The method of claim 22, wherein said pharmaceutical composition comprises one or more copper sensitive compounds such as an omega-3-fatty acid, vitamin C, vitamin E, lutein and/or zeaxanthin.

24. The method of claim 22, wherein said copper is coated with gelatin, a liposome, or a mono- and di-glyceride.

25. The method of claim 22, wherein said copper is copper gluconate, copper oxide, copper carbonate or copper chloride.

26. The method of claim 22, wherein said coating is a zwifterionic phospholipid including but not limited to phosphatidylcholine, phosphatidylserine, phosphatidylethanolamine, spingomyelin and other ceramides, as well as various other zwifterionic phospholipids.

27. The method of claim 22, wherein said copper is a copper cyclodextrin complex.

28. The digestible capsule material of claim 9, wherein said digestible capsule material comprises a gastrointestinal tract digestible capsule material.

29. The digestible capsule material of claim 11, wherein said vitamin C substantially consists of calcium ascorbate.

30. The digestible capsule material of claim 11, wherein said vitamin E substantially consists of d,l alpha tocopheryl acetate.

31. The digestible capsule material of claim 11, wherein said zinc substantially consists of zinc oxide.

32. The digestible capsule material of claim 11, wherein said omega 3 fatty acid substantially consists of Omega-3 acid triglycerides Ph. Eur.
